(19) United States
(12) Patent Application Publication
(10) Pub. No.: US 2010/0226919 A1
(43) Pub. Date: Sep. 9, 2010

(54) Antitumoral Treatments

(75) Inventors: Román Pérez-Soler, Bronx, NY (US); Yi-He Ling, Bronx, NY (US); José Maria Jimeno Donaque, Madrid (ES); Yi-Yu Zou, Bronx, NY (US)

Correspondence Address: KING & SPALDING, 1185 AVENUE OF THE AMERICAS, NEW YORK, NY, 10036-4003, US

(73) Assignees: Pharma Mar, S.A., Madrid (ES); ALBERT EINSTEIN COLLEGE OF MEDICINE OF YESHIVA UNIVERSITY, Bronx, NY (US)

(21) Appl. No.: 12/682,921

(22) PCT Filed: Oct. 17, 2008
(86) PCT No.: PCT/US2008/080309

§ 371 Date: Apr. 14, 2010

Related U.S. Application Data
(60) Provisional application No. 60/981,431, filed on Oct. 19, 2007.

(51) Int. Cl. A61K 38/15; A61P 35/00; A61K 39/395

(52) U.S. Cl. 424/133.1; 514/9; 424/141.1

(57) ABSTRACT

The present invention relates to combinations of PM02734 with EGFR tyrosine kinase inhibitors, and the use of these combinations in the treatment of cancer.

### FIELD OF THE INVENTION

The present invention relates to the combination of PM02734 with other antitumoral agents, in particular with EGFR tyrosine kinase inhibitors, and the use of these combinations in the treatment of cancer.

### BACKGROUND OF THE INVENTION

Cancer develops when cells in a part of the body begin to grow out of control. Although there are many kinds of cancer, they all arise from out-of-control growth of abnormal cells. Cancer cells can invade nearby tissues and can spread through the bloodstream and lymphatic system to other parts of the body. There are several main types of cancer. Carcinoma is a malignant neoplasm, which is an uncontrolled and progressive abnormal growth, arising from epithelial cells. Epithelial cells cover internal and external surfaces of the body, including organs, lining of vessels, and other small cavities. Sarcoma is cancer arising from cells in bone, cartilage, fat, muscle, blood vessels, or other connective or supportive tissue. Leukemia is cancer that arises in blood-forming tissue such as the bone marrow, and causes large numbers of abnormal blood cells to be produced and enter the bloodstream. Lymphoma and multiple myeloma are cancers that arise from cells of the immune system.

In addition, cancer is invasive and tends to infiltrate the surrounding tissues and give rise to metastases. It can spread directly into surrounding tissues and also may be spread through the lymphatic and circulatory systems to other parts of the body.

Many treatments are available for cancer, including surgery and radiation for localised disease, and chemotherapy. However, the efficacy of available treatments for many cancer types is limited, and new, improved forms of treatment showing clinical benefits are needed. This is especially true for those patients presenting with advanced and/or metastatic disease and for patients relapsing with progressive disease after having been previously treated with established therapies which become ineffective or intolerable due to acquisition of resistance or to limitations in administration of the therapies due to associated toxicities.

Since the 1950s, significant advances have been made in the chemotherapeutic management of cancer. Unfortunately, more than 50% of all cancer patients either do not respond to initial therapy or experience relapse after an initial response to treatment and ultimately die from progressive metastatic disease. Thus, the ongoing commitment to the design and discovery of new anticancer agents is critically important.

Chemotherapy, in its classic form, has been focused primarily on killing rapidly proliferating cancer cells by targeting general cellular metabolic processes, including DNA, RNA, and protein biosynthesis. Chemotherapy drugs are divided into several groups based on how they affect specific chemical substances within cancer cells, which cellular activities or processes the drug interferes with, and which specific phases of the cell cycle the drug affects. The most commonly used types of chemotherapy drugs include: DNA-alkylating drugs (such as cyclophosphamide, ifosfamide, cisplatin, carboplatin, dacarbazine), antimetabolites (5-fluorouracil, capecitabine, 6-mercaptopurine, methotrexate, gemcitabine, cytarabine, fludarabine), mitotic inhibitors (such as paclitaxel, docetaxel, vinblastine, vincristine), anthracyclines (such as daunorubicin, doxorubicin, epirubicin, idarubicin, mitoxantrone), topoisomerase I and II inhibitors (such as topotecan, irinotecan, etoposide, teniposide), and hormone therapy (such as tamoxifen, flutamide).

The ideal antitumor drug would kill cancer cells selectively, with a wide index relative to its toxicity towards non-cancer cells and it would also retain its efficacy against cancer cells, even after prolonged exposure to the drug. Unfortunately, none of the current chemotherapies with these agents posses an ideal profile. Most posses very narrow therapeutic indexes and, in addition, cancerous cells exposed to slightly sublethal concentrations of a chemotherapeutic agent may develop resistance to such an agent, and quite often cross-resistance to several other antitumor agents.

PM02734 ((4S)-MeHex-D-Val-L-Thr-L-Val-D-Val-D-Pro-L-Orn-D-allo-Ile-cyclo(D-allo-Thr-D-allo-Ile-D-Val-L-Phe-Z-Dhb-L-Val)) is a novel synthetic depsipeptide related to the family of kahalalide compounds. This compound is the subject of WO 2004/035613 and has the following structure:

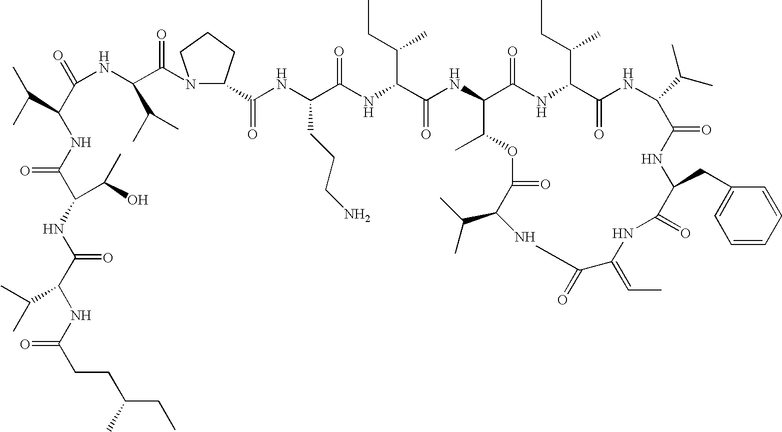

Kahalalide compounds are cyclic depsipeptides which were originally isolated from a Hawaiian herbivorous marine species of mollusk, Elysia rufescens, and its diet, the green alga Briopsis sp. Kahalalides A-G were described by Hamann et al. (J. Am. Chem. Soc. 1993, 115, 5825-5826 and J. Org. Chem. 1996, 61, 6594-6600) and many of them show activity against cancer and AIDS-related opportunistic infections. Some other natural kahalalide compounds have been also disclosed such as Kahalalide H and J by Scheuer et al. (J. Nat. Prod. 1997, 60, 562-567), Kahalalide 0 by Scheuer et al. (J. Nat. Prod. 2000, 63(1), 152-154), Kahalalide K by Kan et al. (J. Nat. Prod. 1999, 62(8), 1169-1172).

Of the kahalalide compounds of natural origin, Kahalalide F is the most promising because of its antitumoral activity. EP 610.078 reports that early preclinical in vitro screening studies identified micromolar activity of Kahalalide F against mouse leukemia (P388) and two human solid tumors: non-small cell lung (A549) and colon (HT-29). The primary mechanism of Kahalalide F action has not been identified yet, however it has been found that Kahalalide F is an NCI-COMPARE compound that induces sub G1 cell-cycle arrest and cytotoxicity independently of MDR, Her2, P53, and blc-2 (Janmaat et al. Proceedings of the 2^{nd} International Symposium on Signal Transduction Modulators in Cancer Therapy: 23-25 Oct., Amsterdam 2003: 60 (Abst. B02)). The COMPARE analysis in a panel of 60 human cancer cell lines genetically and molecularly characterized for cell proliferation pathways has included Kahalalide F in the list of new chemical entities that interact with the Erb/Her-neu pathway (Wosikowski et al. J. Natl. Cancer Inst. 1997, 89, 1505-1515). Sensitivity to Kahalalide F significantly correlated with baseline expression levels of ErbB3 (HER3), but not of other ErbB receptors, in a panel of established cell lines from different origins. Furthermore, the downstream PI3K/Akt pathway coupled to ErbB3 receptor is also affected by Kahalalide F treatment. Kahalalide F decreases phosphorylated Akt levels and this reduction is associated with cytotoxicity in Kahalalide F-sensitive cell lines (Janmaat et al. Mol Pharmacol 2005, 68, 502-510).

PM02734 has showed significant improved efficacy in in vivo cancer models with respect to those activities observed with kahalalide compounds of natural origin, and specifically with Kahalalide F. PM02734 has demonstrated in vitro antitumor activity against a broad spectrum of tumor types such as leukemia, melanoma, breast, colon, ovary, pancreas, lung, and prostate, and has shown significant in vivo activity in xenografted murine models using human tumor cell types such as breast, prostate, and melanoma.

More information on PM02734 and other kahalalide compounds, in particular Kahalalide F and analogs thereof, their uses, formulations and synthesis can be found in the patent applications EP 610.078, WO 2004/035613, WO 01/58934, WO 2005/023846, WO 2004/075910, WO 03/033012, WO 02/36145, and WO 2005/103072. We incorporate by specific reference the content of each of these EP and PCT texts.

Since cancer is a leading cause of death in animals and humans, several efforts have been and are still being undertaken in order to obtain an antitumor therapy active and safe to be administered to patients suffering from a cancer. The problem to be solved by the present invention is to provide antitumor therapies that are useful in the treatment of cancer.

### SUMMARY OF THE INVENTION

We have established that PM02734 potentiates other anticancer agents, in particular those from the group of EGFR tyrosine kinase inhibitors, and therefore they can be successfully used in combination therapy for the treatment of cancer. Thus, this invention is directed to pharmaceutical compositions, kits, methods for the treatment of cancer using these combination therapies and uses of PM02734 in the manufacture of a medicament for combination therapy.

In accordance with one aspect of this invention, we provide effective combination therapies for the treatment of cancer based on PM02734 and using EGFR tyrosine kinase inhibitors.

In another embodiment the invention encompasses a method of treating cancer comprising administering to a patient in need of such treatment a therapeutically effective amount of PM02734, or a pharmaceutically acceptable salt thereof, and a therapeutically effective amount of an EGFR tyrosine kinase inhibitor, or a pharmaceutically acceptable salt thereof, administered prior, during, or after administering PM02734. The two drugs may form part of the same composition, or be provided as a separate composition for administration at the same time or at a different time.

In another aspect the invention encompasses a method of increasing the therapeutic efficacy of an EGFR tyrosine kinase inhibitor in the treatment of cancer, which comprises administering to a patient in need thereof a therapeutically effective amount of PM02734, or a pharmaceutically acceptable salt thereof. PM02734 is administered prior, during, or after administering the EGFR tyrosine kinase inhibitor.

In another embodiment the invention encompasses the use of PM02734, or a pharmaceutically acceptable salt thereof, for the manufacture of a medicament for the treatment of cancer, in combination therapy with an EGFR tyrosine kinase inhibitor.

In a related embodiment the invention encompasses the use of an EGFR tyrosine kinase inhibitor, or a pharmaceutically acceptable salt thereof, for the manufacture of a medicament for the treatment of cancer, in combination therapy with PM02734.

In a further aspect the invention encompasses a pharmaceutical composition comprising PM02734, or a pharmaceutically acceptable salt thereof, and/or an EGFR tyrosine kinase inhibitor, or a pharmaceutically acceptable salt thereof, to be used in combination therapy for the treatment of cancer.

The invention also encompasses a kit for use in the treatment of cancer which comprises a dosage form of PM02734, or a pharmaceutically acceptable salt thereof, and/or a dosage form of an EGFR tyrosine kinase inhibitor, or a pharmaceutically acceptable salt thereof, and instructions for the use of both drugs in combination.

In one preferred aspect, the present invention is concerned with synergistic combinations of PM02734, or a pharmaceutically acceptable salt thereof, with EGFR tyrosine kinase inhibitors, or a pharmaceutically acceptable salt thereof.

### BRIEF DESCRIPTION OF THE FIGURES

FIG. 1. Relationship between PM02734-induced cytotoxicity and expression of ErbB family proteins in human NSCLC cell lines. Data represent the mean±S.D. of three independent experiments.

FIG. 2. Effect of PM02734 on cell-cycle progression and apoptosis in H322 cells.

FIG. 3. Synergistic effect between PM02734 and erlotinib in human H322 and A549 NSCLC cell lines. Data represent the mean±S.D. of three independent experiments.

FIG. 4. Synergistic effect between PM02734 and erlotinib in human H1299 and H460 NSCLC cell lines. Data represent the mean±S.D. of three independent experiments.

FIG. 5. Cell growth inhibition by schematic exposure to PM02734 and erlotinib in H322 and H1299 cells. (A), Schematic representation of different exposure schedules of PM02734 and erlotinib. (B), Cell survival. Data represent the mean of two independent of experiments. PM: PM02734; E: Erlotinib.

FIG. 6. Effects of PM02734, erlotinib, and combination of both agents on the activation of EGFR and its related signal pathways in H322 cells.

FIG. 7. Efficacy of PM02734 combined with erlotinib in A549 subcutaneous xenograft model.

FIG. 8. Efficacy of PM02734 combined with erlotinib in A549 intravenous xenograft model. (A), 4.2×10⁶ tumor cells were i.v. inoculated per mouse. (B), 8.4×10⁶ tumor cells were i.v. inoculated per mouse.

### DETAILED DESCRIPTION OF THE INVENTION

In order to study the possible potentiation of EGFR tyrosine kinase inhibitors with PM02734 we initiated a systematic study firstly to determine the antitumor effect of PM02734 against certain tumor cells, secondly to determine the existence of any interrelation between PM02734 and EGF receptors, thirdly to determine the existence of any possible synergism between the effect of PM02734 and the effect of EGFR tyrosine kinase inhibitors when administered in combination. As a general conclusion we found that the antitumor activity of EGFR tyrosine kinase inhibitors is greatly enhanced in combination with PM02734. Thus, the present invention is directed to providing an efficacious treatment of cancer based on the combination of PM02734 with an EGFR tyrosine kinase inhibitor.

By “cancer” it is meant to include tumors, neoplasias, and any other malignant tissue or cells.

In another aspect, the invention relates to synergistic combinations employing PM02734, or a pharmaceutically acceptable salt thereof, and EGFR tyrosine kinase inhibitors, or a pharmaceutically acceptable salt thereof. An indication of synergy can easily be obtained by testing combinations and analyzing the results, for example by the Chou-Talalay method. Reference is made to Example 4 to illustrate this point.

The term “combination” as used throughout the specification, is meant to encompass the administration of the therapeutic agents in the same or separate pharmaceutical formulations, and at the same time or at different times. If the therapeutic agents are administered at different times they should be administered sufficiently close in time to provide for the synergistic response to occur.

In another aspect, the invention is directed to the use of PM02734, or a pharmaceutically acceptable salt thereof, for the manufacture of a medicament for an effective treatment of cancer by combination therapy employing PM02734, or a pharmaceutically acceptable salt thereof, with an EGFR tyrosine kinase inhibitor, or a pharmaceutically acceptable salt thereof.

In a related aspect, the invention is directed to the use of an EGFR tyrosine kinase inhibitor, or a pharmaceutically acceptable salt thereof, for the manufacture of a medicament for an effective treatment of cancer by combination therapy employing an EGFR tyrosine kinase inhibitor, or a pharmaceutically acceptable salt thereof, with PM02734, or a pharmaceutically acceptable salt thereof.

In a further aspect, the present invention is directed to a method of treating cancer comprising administering to a patient in need of such treatment a therapeutically effective amount of PM02734, or a pharmaceutically acceptable salt thereof, in combination with a therapeutically effective amount of an EGFR tyrosine kinase inhibitor, or a pharmaceutically acceptable salt thereof.

The invention also provides a method of treating cancer comprising administering a therapeutically effective amount of an EGFR tyrosine kinase inhibitor, or a pharmaceutically acceptable salt thereof, in combination with a therapeutically effective amount of PM02734, or a pharmaceutically acceptable salt thereof.

As mentioned above, PM02734 ((4S)-MeHex-D-Val-L-Thr-L-Val-D-Val-D-Pro-L-Orn-D-allo-Ile-cyclo(D-allo-Thr-D-allo-Ile-D-Val-L-Phe-Z-Dhb-L-Val)) is a synthetic depsipeptide with the following structure:

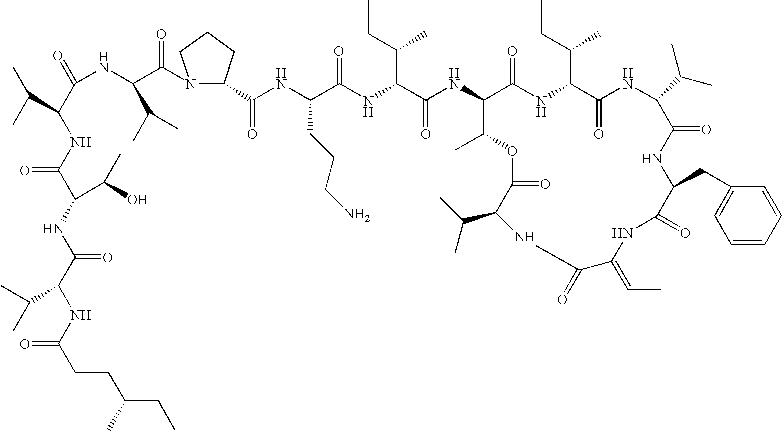

The term “PM02734” is intended here to cover any pharmaceutically acceptable salt, ester, solvate, hydrate, prodrug, or any other compound which, upon administration to the patient is capable of providing (directly or indirectly) the compound as described herein. However, it will be appreciated that non-pharmaceutically acceptable salts also fall within the scope of the invention since these may be useful in the preparation of pharmaceutically acceptable salts. The preparation of salts, esters, solvates, hydrates, prodrugs, and derivatives can be carried out by methods known in the art.

For instance, pharmaceutically acceptable salts of PM02734 are synthesized from the parent compound, which contains a basic moiety, by conventional chemical methods. Generally, such salts are, for example, prepared by reacting the free base form of this compound with a stoichiometric amount of the appropriate acid in water or in an organic solvent or in a mixture of the two. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methanesulphonate and p-toluenesulphonate. Trifluoroacetate salts of PM02734 are specially preferred.

In addition, PM02734 may be in crystalline form either as free compound or as solvates (e.g. hydrates) and it is intended that both forms are within the scope of the present invention. Methods of solvation are generally known within the art.

Any compound that is a prodrug of PM02734 is within the scope and spirit of the invention. The term “prodrug” is used in its broadest sense and encompasses those derivatives that are converted in vivo to PM02734. The prodrug can hydrolyze, oxidize, or otherwise react under biological conditions to provide PM02734. Such derivatives would readily occur to those skilled in the art, and include, for example, compounds where a free hydroxy group is converted into an ester derivative.

Any compound referred to herein is intended to represent such specific compound as well as certain variations or forms. In particular, compounds referred to herein may have asymmetric centres and therefore exist in different enantiomeric forms. All optical isomers and stereoisomers of the compounds referred to herein, and mixtures thereof, are considered within the scope of the present invention. Thus any given compound referred to herein is intended to represent any one of a racemate, one or more enantiomeric forms, one or more diastereomeric forms, one or more atropisomeric forms, and mixtures thereof. Particularly, the compounds of the present invention may include enantiomers depending on their asymmetry or diastereoisomers. Stereoisomerism about the double bond is also possible, therefore in some cases the molecule could exist as (E)-isomer or (Z)-isomer. If the molecule contains several double bonds, each double bond will have its own stereoisomerism, that could be the same or different than the stereoisomerism of the other double bonds of the molecule. The single isomers and mixtures of isomers fall within the scope of the present invention.

Furthermore, compounds referred to herein may exist as geometric isomers (i.e., cis and trans isomers), as tautomers, or as atropisomers. Specifically, the term tautomer refers to one of two or more structural isomers of a compound, that exist in equilibrium and are readily converted from one isomeric form to another. Common tautomeric pairs are amine-imine, amide-imide, keto-enol, lactam-lactim, etc. Additionally, any compound referred to herein is intended to represent hydrates, solvates, and polymorphs, and mixtures thereof when such forms exist in the medium. In addition, compounds referred to herein may exist in isotopically-labelled forms. All geometric isomers, tautomers, atropisomers, hydrates, solvates, polymorphs, and isotopically labelled forms of the compounds referred to herein, and mixtures thereof, are considered within the scope of the present invention.

PM02734 for use in accordance of the present invention may be prepared following a synthetic process such as those disclosed in WO 2004/035613, WO 2005/103072, WO 01/58934, and WO 2005/023846, which are incorporated herein by reference.

Pharmaceutical compositions of PM02734, or of a pharmaceutically acceptable salt thereof, that can be used include solutions, suspensions, emulsions, lyophilized compositions, etc., with suitable excipients for intravenous administration. For further guidance on pharmaceutical compositions of PM02734, or a pharmaceutically acceptable salt thereof, see for example WO 2004/035613 which is incorporated herein by reference.

Administration of PM02734, or a pharmaceutically acceptable salt thereof, or pharmaceutical compositions comprising the compound is preferably by intravenous infusion. Infusion times of up to 72 hours can be used, more preferably 1 to 24 hours, with either about 1 hour or about 3 hours most preferred. Short infusion times which allow treatment to be carried out without an overnight stay in hospital are especially desirable. However, infusion may be around 24 hours or even longer if required.

Preferably the administration of PM02734 is performed in cycles. In a preferred application method an intravenous infusion of PM02734 is given to the patients the first week of each cycle and the patients are allowed to recover for the remainder of the cycle. The preferred duration of each cycle is of either 1, 3, or 4 weeks. Multiple cycles can be given as needed. In an alternative dosing protocol, PM02734 is administered for say about 1 hour for 5 consecutive days every 3 or 4 weeks. Other protocols can be devised as variations. For further guidance on PM02734 administration and dosages, see for example WO 2004/035613 which is incorporated herein by reference.

Several approaches to target protein tyrosine kinases have been developed. Tyrosine kinases play a critical role in the modulation of growth factor signaling. Activated forms of these enzymes can cause increases in tumor cell proliferation and growth, induce antiapoptotic effects, and promote angiogenesis and metastasis. In addition to activation by growth factors, protein kinase activation by somatic mutation is a common mechanism of tumor genesis. Because all of these effects are initiated by receptor tyrosine kinase activation, they are key targets for inhibitors.

A subclass of the receptor tyrosine kinase superfamily consists of the ErbB or epidermal growth factor (EGF) receptors and comprises four members: EGFR/ErbB1, ErbB2 (HER2), ErbB3 (HER3) and ErbB4 (HER4). The ERBB or EGF receptors are aberrantly activated in a wide range of human tumors, and as such they are excellent candidates for selective anticancer therapies. Several antibodies directed against the extracellular domain of ERBBs and small-molecule tyrosine-kinase inhibitors that target the kinase domain are in clinical use or at advanced developmental stages.

Preferred EGFR tyrosine kinase inhibitors to be used in combination with PM02734, or a pharmaceutically acceptable salt thereof, are erlotinib, gefitinib, canertinib, lapatinib, cetuximab, matuzumab, zalutumumab, and panitumumab, or a pharmaceutically acceptable salt thereof; being erlotinib, gefitinib, canertinib, and lapatinib, or a pharmaceutically acceptable salt thereof, specially preferred; and being erlotinib, or a pharmaceutically acceptable salt thereof, one of the most preferred. Information about these drugs is available on the extensive literature on them (see for example Hynes et al. Nature Reviews, 2005, 5, 341-354; Arora et al. JPET, 2005, 315, 971-979; and Steeghs et al. Ann. Surg. Oncol. 2007, 14(2), 942-953).

Erlotinib is a quinazolinamine with the following structural formula:

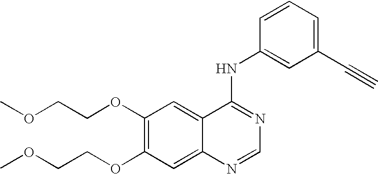

This drug is being marketed in the form of its hydrochloride salt with the trade name Tarceva®. This drug is currently indicated for the treatment of certain cancer, specifically for non-small cell lung cancer (NSCLC) and pancreatic cancer. In addition, erlotinib is recommended to be administered orally once daily at a dose of 100 mg or 150 mg a day depending on the indication. Information about this drug is available on the website www.tarceva.com and the extensive literature on erlotinib. Although the mechanism of clinical antitumor action of erlotinib is currently not fully characterized, it is known that it inhibits the intracellular phosphorylation of tyrosine kinase associated with the EGFR. Specificity of inhibition with regard to other tyrosine kinase receptors has not been fully characterized.

Other preferred EGFR tyrosine kinase inhibitors to be used in combination with PM02734 are those disclosed in U.S. Pat. No. 5,457,105, U.S. Pat. No. 5,770,599, U.S. Pat. No. 5,747,498, U.S. Pat. No. 6,344,455, U.S. Pat. No. 6,391,874, U.S. Pat. No. 6,713,485, U.S. Pat. No. 6,727,256, U.S. Pat. No. 6,900,221, U.S. Pat. No. 7,157,466, U.S. Pat. No. 4,943,533, U.S. Pat. No. 5,558,864, U.S. Pat. No. 5,891,996, and U.S. Pat. No. 6,235,883. These patents are incorporated herein in full by specific reference. In particular, for the present invention, we preferred that the EGFR tyrosine kinase inhibitor is a compound or a monoclonal antibody claimed in any of the U.S. Pat. No. 5,457,105, U.S. Pat. No. 5,770,599, U.S. Pat. No. 5,747,498, U.S. Pat. No. 6,344,455, U.S. Pat. No. 6,391,874, U.S. Pat. No. 6,713,485, U.S. Pat. No. 6,727,256, U.S. Pat. No. 6,900,221, U.S. Pat. No. 7,157,466, and U.S. Pat. No. 4,943,533, U.S. Pat. No. 5,558,864, U.S. Pat. No. 5,891,996, and U.S. Pat. No. 6,235,883.

PM02734, or a pharmaceutically acceptable salt thereof, and the EGFR tyrosine kinase inhibitor, or a pharmaceutically acceptable salt thereof, may be provided as separate medicaments for administration at the same time or at different times. Preferably, PM02734, or a pharmaceutically acceptable salt thereof, and the EGFR tyrosine kinase inhibitor, or a pharmaceutically acceptable salt thereof, are provided as separate medicaments for administration at different times. When administered separately and at different times, either PM02734, or a pharmaceutically acceptable salt thereof, or the EGFR tyrosine kinase inhibitor, or a pharmaceutically acceptable salt thereof, may be administered first. In addition, both drugs can be administered in the same day or at different days, and they can be administered using the same schedule or at different schedules during the treatment cycle. Thus, the pharmaceutical compositions of the present invention may comprise all the components (drugs) in a single pharmaceutically acceptable formulation. Alternatively, the components may be formulated separately and administered in combination with one another. Various pharmaceutically acceptable formulations well known to those of skill in the art can be used in the present invention.

Preferably, a combination of PM02734, or a pharmaceutically acceptable salt thereof, and a EGFR tyrosine kinase inhibitor, or a pharmaceutically acceptable salt thereof, can be used in any suitable formulation for combined or separate intravenous administration. The intravenous formulations of the combination may include solutions, suspensions, emulsions, lyphilised compositions and the like. However, selection of an appropriate formulation for use in the present invention can be performed routinely by those skilled in the art based upon the mode of administration and the solubility characteristics of the components of the composition.

The correct dosage of the compounds of the combination will vary according to the particular formulation, the mode of application, and the particular site, host and tumour being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

In another aspect, the present invention is directed to a kit for administering PM02734 in combination with an EGFR tyrosine kinase inhibitor in the treatment of cancer, comprising a supply of PM02734, or a pharmaceutically acceptable salt, in dosage units for at least one cycle, and printed instructions for the use of both drugs in combination.

In a related aspect, the present invention is directed to a kit for administering an EGFR tyrosine kinase inhibitor in combination with PM02734 in the treatment of cancer, comprising a supply of an EGFR tyrosine kinase inhibitor, or a pharmaceutically acceptable salt thereof, in dosage units for at least one cycle, and printed instructions for the use of both drugs in combination.

In a related aspect, the present invention is directed to a kit for administering PM02734 in combination with an EGFR tyrosine kinase inhibitor in the treatment of cancer, comprising a supply of PM02734, or a pharmaceutically acceptable salt thereof, in dosage units for at least one cycle, a supply of the EGFR tyrosine kinase inhibitor, or a pharmaceutically acceptable salt thereof, in dosage units for at least one cycle, and printed instructions for the use of both drugs in combination.

In another aspect, the present invention also provides a pharmaceutical composition comprising PM02734, or a pharmaceutically acceptable salt thereof, and a pharmaceutically acceptable carrier, for use in combination with an EGFR tyrosine kinase inhibitor in the treatment of cancer.

In a further aspect, the present invention also provides a pharmaceutical composition comprising an EGFR tyrosine kinase inhibitor, or a pharmaceutically acceptable salt thereof, and a pharmaceutically acceptable carrier, for use in combination with PM02734 in the treatment of cancer.

In addition, the present invention also provides a pharmaceutical composition comprising PM02734, or a pharmaceutically acceptable salt thereof, an EGFR tyrosine kinase inhibitor, or a pharmaceutically acceptable salt thereof, and a pharmaceutically acceptable carrier, for use in the treatment of cancer.

In another aspect, the invention further provides for the use of PM02734, or a pharmaceutically acceptable salt thereof, in the preparation of a composition for use in combination with an EGFR tyrosine kinase inhibitor in the treatment of cancer.

In a related aspect, the invention further provides for the use of an EGFR tyrosine kinase inhibitor, or a pharmaceutically acceptable salt thereof, in the preparation of a composition for use in combination with PM02734 in the treatment of cancer.

And in a further aspect, the invention also provides for the use of PM02734, or a pharmaceutically acceptable salt thereof, and an EGFR tyrosine kinase inhibitor, or a pharmaceutically acceptable salt thereof, in the preparation of a composition for use in the treatment of cancer.

In another aspect, the invention further provides for the use of PM02734, or a pharmaceutically acceptable salt thereof, for the manufacture of a medicament for the treatment of cancer, in combination therapy with an EGFR tyrosine kinase inhibitor.

In a related aspect, the invention further provides for the use of an EGFR tyrosine kinase inhibitor, or a pharmaceutically acceptable salt thereof, for the manufacture of a medicament for the treatment of cancer, in combination therapy with PM02734.

In a related aspect, the invention further provides for the use of PM02734, or a pharmaceutically acceptable salt thereof in combination with an EGFR tyrosine kinase inhibitor, or a pharmaceutically acceptable salt thereof, for the manufacture of a medicament for the treatment of cancer.

In another aspect, the invention further provides for the use of PM02734, or a pharmaceutically acceptable salt thereof, for the treatment of cancer, in combination therapy with an EGFR tyrosine kinase inhibitor.

In a related aspect, the invention further provides for the use of an EGFR tyrosine kinase inhibitor, or a pharmaceutically acceptable salt thereof, for the treatment of cancer, in combination therapy with PM02734.

In another aspect, the invention further provides for the use of PM02734, or a pharmaceutically acceptable salt thereof, in combination with an EGFR tyrosine kinase inhibitor, or a pharmaceutically acceptable salt thereof, for the treatment of cancer.

In another aspect, the invention further provides for the use of PM02734, or a pharmaceutically acceptable salt thereof, as a medicament, in combination therapy with an EGFR tyrosine kinase inhibitor.

In a related aspect, the invention further provides for the use of an EGFR tyrosine kinase inhibitor, or a pharmaceutically acceptable salt thereof, as a medicament, in combination therapy with PM02734.

In another aspect, the invention further provides for the use of PM02734, or a pharmaceutically acceptable salt thereof, in combination with an EGFR tyrosine kinase inhibitor, or a pharmaceutically acceptable salt thereof, as a medicament.

In another aspect, the invention further provides for the use of PM02734, or a pharmaceutically acceptable salt thereof, as a medicament for the treatment of cancer, in combination therapy with an EGFR tyrosine kinase inhibitor, or a pharmaceutically acceptable salt thereof.

In a related aspect, the invention further provides for the use of an EGFR tyrosine kinase inhibitor, or a pharmaceutically acceptable salt thereof, as a medicament for the treatment of cancer, in combination therapy with PM02734, or a pharmaceutically acceptable salt thereof.

In another aspect, the invention further provides for the use of PM02734, or a pharmaceutically acceptable salt thereof, in combination with an EGFR tyrosine kinase inhibitor, or a pharmaceutically acceptable salt thereof, as a medicament for the treatment of cancer.

In another aspect, the invention provides PM02734, or a pharmaceutically acceptable salt thereof, for the treatment of cancer comprising administering a therapeutically effective amount of PM02734, or a pharmaceutically acceptable salt thereof, in combination with a therapeutically effective amount of an EGFR tyrosine kinase inhibitor, or a pharmaceutically acceptable salt thereof.

In a related aspect, the invention further provides an EGFR tyrosine kinase inhibitor, or a pharmaceutically acceptable salt thereof, for the treatment of cancer comprising administering a therapeutically effective amount of an EGFR tyrosine kinase inhibitor, or a pharmaceutically acceptable salt thereof, in combination with a therapeutically effective amount of PM02734, or a pharmaceutically acceptable salt thereof.

In another aspect, the invention provides for the treatment of cancer comprising the administration of therapeutically effective amounts of PM02734, or pharmaceutically acceptable salt thereof, in combination with the administration of therapeutically effective amounts of an EGFR tyrosine kinase inhibitor, or a pharmaceutically salt thereof, wherein the combination may be administered together or separately.

Depending on the type of tumor and the development stage of the disease, the treatments of the invention are useful in promoting tumor regression, in stopping tumor growth and/or in preventing metastasis. In particular, the method of the invention is suited for human patients, especially those who are relapsing or refractory to previous chemotherapy. First line therapy is also envisaged.

Preferably, the combination of PM02734, or a pharmaceutically acceptable salt thereof, with an EGFR tyrosine kinase inhibitor, or a pharmaceutically acceptable salt thereof, is used for the treatment of leukemia, melanoma, breast cancer, colon cancer, colorectal cancer, ovarian cancer, gynecological cancer, renal cancer, head & neck carcinoma, esophageal cancer, pancreatic cancer, lung cancer, cervix cancer, liver cancer, and prostate cancer. Specially preferred is the use of the combination for the treatment of lung cancer, breast cancer, and pancreas cancer. Most preferably the combination is used for the treatment of lung cancer, specifically non-small cell lung cancer (NSCLC).

In one embodiment, cancer cells are contacted, or otherwise treated, with a combination of PM02734, or a pharmaceutically acceptable salt thereof, and an EGFR tyrosine kinase inhibitor, or a pharmaceutically acceptable salt thereof. The cancer cells are preferably human and may include carcinoma cells, sarcoma cells, leukemia cells, lymphoma cells and myeloma cells. More preferably, the cancer cells may include leukemia cells, melanoma cells, breast cancer cells, colon cancer cells, colorectal cancer cells, ovarian cancer cells, gynecological cancer cells, renal cancer cells, head & neck carcinoma cells, esophageal cancer cells, pancreatic cancer cells, lung cancer cells, cervix cancer cells, liver cancer cells, and prostate cancer cells. In particular, the cancer cells may include human non small cell lung cancer cells. In addition, the combination may provide a synergistic inhibitory effect against cancer cells, particularly against human non small cell lung cancer cells.

For example, the cancer cells may be in culture and the combination may be administered in vitro. The combination may be delivered together or separately. A lower level of proliferation or survival of the contacted cancer cells in culture compared to the non-contacted cancer cells in culture suggests that the combination of PM02734, or a pharmaceutically acceptable salt thereof, and an EGFR tyrosine kinase inhibitor, or a pharmaceutically acceptable salt thereof, may be effective for treating a patient with that particular type of cancer.

In addition, appropriate in vitro techniques can be performed routinely by those skilled in the art to determine the mechanism of action for a combination of PM02734, or a pharmaceutically acceptable salt thereof, and an EGFR tyrosine kinase inhibitor, or a pharmaceutically acceptable salt thereof. For example, flow cytometry may be utilized to investigate whether the combination cause disturbance of cell-cycle progression and/or increase apoptosis. Western blot analysis using suitable antibodies may also be utilized to determine the mechanism of action.

In another aspect, the invention provides for a method for inhibiting the growth of cancer cells comprising contacting said cancer cells with an effective amount of PM02734, or a pharmaceutically acceptable salt thereof, in combination with an EGFR tyrosine kinase inhibitor.

In a related aspect, the invention provides for a method for inhibiting the growth of cancer cells comprising contacting said cancer cells with an effective amount of an EGFR tyrosine kinase inhibitor, or a pharmaceutically acceptable salt thereof, in combination with PM02734.

In a related aspect, the invention provides for a method for inhibiting the growth of cancer cells comprising contacting said cancer cell with an effective combination of PM02734, or a pharmaceutically acceptable salt thereof, and an EGFR tyrosine kinase inhibitor, or a pharmaceutically acceptable salt thereof, together or separately.

In another aspect, the invention provides for a method for inhibiting the growth of cancer cells comprising contacting said cancer cell with a synergistic combination of PM02734, or a pharmaceutically acceptable salt thereof, and an EGFR tyrosine kinase inhibitor, or a pharmaceutically acceptable salt thereof, together or separately, wherein said combination provides improved inhibition against cancer cell growth as compared to (i) PM02734, or a pharmaceutically acceptable salt thereof, in the absence of an EGFR tyrosine kinase inhibitor or (ii) an EGFR tyrosine kinase inhibitor, or pharmaceutically acceptable salt thereof, in the absence of PM02734.

In another aspect, the invention provides for a pharmaceutical composition comprising an effective amount of PM02734, or a pharmaceutically acceptable salt thereof, for use in combination with an EGFR tyrosine kinase inhibitor for inhibiting the growth of cancer cells.

In a related aspect, the invention provides for a pharmaceutical composition comprising an effective amount of an EGFR tyrosine kinase inhibitor, or a pharmaceutically acceptable salt thereof, for use in combination with PM02734 for inhibiting the growth of cancer cells.

In a related aspect, the invention provides for a pharmaceutical composition comprising an effective combination of PM02734, or a pharmaceutically acceptable salt thereof, and an EGFR tyrosine kinase inhibitor, for inhibiting the growth of cancer cells.

In another aspect, the invention provides for a pharmaceutical composition comprising a synergistic combination of PM02734, or a pharmaceutically acceptable salt thereof, and an EGFR tyrosine kinase inhibitor, or a pharmaceutically acceptable salt thereof, for inhibiting the growth of cancer cells, wherein said combination provides improved inhibition against cancer cell growth as compared to (i) PM02734, or a pharmaceutically acceptable salt thereof, in the absence of an EGFR tyrosine kinase inhibitor or (ii) an EGFR tyrosine kinase inhibitor, or pharmaceutically acceptable salt thereof, in the absence of PM02734.

In another aspect, the invention provides for a method for disrupting cell-cycle progression of cancer cells comprising contacting said cancer cells with an effective amount of PM02734, or a pharmaceutically acceptable salt thereof, in combination with an EGFR tyrosine kinase inhibitor.

In a related aspect, the invention provides for a method for disrupting cell-cycle progression of cancer cells comprising contacting said cancer cells with an effective amount of an EGFR tyrosine kinase inhibitor, or a pharmaceutically acceptable salt thereof, in combination with PM02734.

In a related aspect, the invention provides for a method for disrupting cell-cycle progression of cancer cells comprising contacting said cancer cell with an effective combination of PM02734, or a pharmaceutically acceptable salt thereof, and an EGFR tyrosine kinase inhibitor, or a pharmaceutically acceptable salt thereof, together or separately.

In another aspect, the invention provides for a method for increasing apoptosis of cancer cells comprising contacting said cancer cells with an effective amount of PM02734, or a pharmaceutically acceptable salt thereof, in combination with an EGFR tyrosine kinase inhibitor.

In a related aspect, the invention provides for a method for increasing apoptosis of cancer cells comprising contacting said cancer cells with an effective amount of an EGFR tyrosine kinase inhibitor, or a pharmaceutically acceptable salt thereof, in combination with PM02734.

In a related aspect, the invention provides for a method for increasing apoptosis of cancer cells comprising contacting said cancer cell with an effective combination of PM02734, or a pharmaceutically acceptable salt thereof, and an EGFR tyrosine kinase inhibitor, or a pharmaceutically acceptable salt thereof, together or separately.

In another aspect, the invention provides for a method for inhibiting AKT in cancer cells comprising contacting said cancer cells with an effective amount of PM02734, or a pharmaceutically acceptable salt thereof, in combination with an EGFR tyrosine kinase inhibitor.

In a related aspect, the invention provides for a method for inhibiting AKT in cancer cells comprising contacting said cancer cells with an effective amount of an EGFR tyrosine kinase inhibitor, or a pharmaceutically acceptable salt thereof, in combination with PM02734.

In a related aspect, the invention provides for a method for inhibiting AKT in cancer cells comprising contacting said cancer cell with an effective combination of PM02734, or a pharmaceutically acceptable salt thereof, and an EGFR tyrosine kinase inhibitor, or a pharmaceutically acceptable salt thereof, together or separately.

The following example further illustrates the invention. It should not be interpreted as a limitation of the scope of the invention.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

### EXAMPLES

### Example 1

### Determination of PM02734-Induced Cytotoxicity in Human NSCLC Cell Lines

Nine human NSCLC cell lines that were characterized for EGFR, Ras, and p53 gene expression and sensitivity to a variety of antitumor agents (paclitaxel, premetrexed, erlotinib, etc.) were used as models to examine the cell sensitivity to PM02734 trifluoroacetate salt. These NSCLC cell lines are the following: H322, A549, H661, H1299, H1975 with L858R and T790M EGFR double mutation, H358, H460, H1650 with L858R EGFR mutation, and H3255 with L858L EGFR mutation. All NSCLC cell lines used in this investigation except H3255 cell line were obtained from American Type Culture Collection (ATCC, Manassas, Va.) (Li et al. Clin. Cancer Res. 2007, 13 (11), 3413-3422). H3255 cell line is a gift from Dr. Pasi A. Jänne (Dana-Farber Cancer Institute, Boston Mass.).

All cell lines were grown in RPMI1640 medium with 10% fetal bovine serum and maintained at 37° C. in a humidified atmosphere of 95% air and 5% CO₂. Exponentially growing cells (1×10⁵/ml) were plated in 96-well plates and allowed to attach overnight. Cells were exposed to various concentrations of PM02734 trifluoroacetate salt at 37° C. for 72 hours. After treatment, the cell survival fraction was assessed by the reduction of tetrazolium bromide (MTT) assay or the cell viability was assessed by cell count using trypan blue exclusion (Ling et al. Cancer Res. 1993, 53 (7), 1583-1589). IC₅₀ value resulting from 50% cell growth inhibition was calculated graphically.

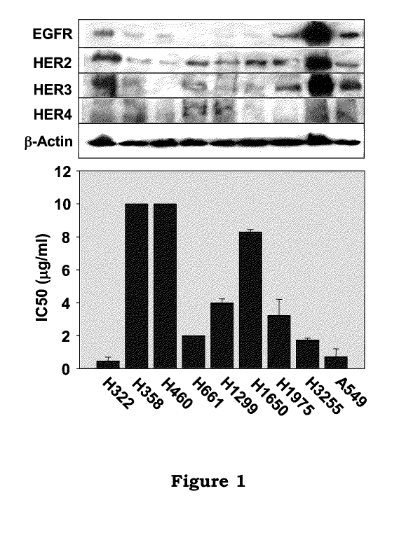

Of nine cell lines tested, four cell lines (H322, A549, H3255-EGFR mutant-, and H661) were highly sensitive to PM02734 trifluoroacetate salt, with IC₅₀ values of 0.3-1.25 μM, two cell lines (H1299 and H1975-double EGFR mutant-) had an intermediate sensitivity, with IC₅₀ values of 2-2.8 μM, and the remaining cell lines (H358, H460, and H1650-EGFR mutant-) were resistant to PM02734 trifluoroacetate salt with IC₅₀ values>5 μM (FIG. 1).

Interestingly, two of the PM02734 sensitive cell lines (H322 and H3255) were the only two cell lines sensitive to erlotinib.

### Example 2

### Correlation Between Expression of EGFR Family Receptors and Cell Sensitivity to PM02734

Since a broad spectrum of sensitivity to PM02734 was found in different NSCLC cell lines, we investigated whether the expression of endogenous EGFR family receptors correlated with cell sensitivity to PM02734.

Exponentially growing cells were harvested by trypsinization. After centrifugation, the cell pellet was divided into two parts. One aliquot was prepared for the determination of basal levels of EGFR family receptors and phosphorylated EGFR family receptors by immunoblotting analysis (further information about immunoblot analysis is provided in Ling et al. Mol. Pharmacol. 2007, 72, 248-258) using corresponding antibodies (Cell Signaling, Beverly, Mass.). The other aliquot was prepared for the measurement of the basal levels of mRNA of EGFR family receptors using real time RT-PCR. To quantitatively measure mRNA expression, total cellular RNA was isolated from tested cell lines by using phenol/chloroform extraction technique. All primers were designed and tested for their specificity using the primer express v1.5 (Applied Biosystems, Foster City, Calif.). The primers specific for EGFR (erbB-1) were 5′-CCACCTGTGCCATCCAAACT (SEQ ID NO.:1) and 5′-GGCGATGGACGGGATCTT (SEQ ID NO.:2); for erbB-2 were 5′-AGCCTTGCCCATCAACTG (SEQ ID NO.:3) and 5′-AATGCCAACCACCGCAGA (SEQ ID NO.:4); and for erbB-3 were 5′-TCCTGGCCGCCCCACATGCACAAC-3′ (SEQ ID NO.:5) and 5′-GTCACATTTGCCCTCTGCCA-3′ (SEQ ID NO.:6). β-Actin RNA (5′-CATGGGTCAGAAGGATTCCT (SEQ ID NO.:7) and 5′-CATTGTAGAAGGTGTGGTGC) (SEQ ID NO.:8) was used as an internal standard. All assays were performed using duplicated samples of reverse transcriptase product. The mRNA expression of EGFR, erbB-2, and erbB-3 was normalized using the dCt=[Ct(EGFR or erbB-2)-Ct((β-actin)] method (Livak KJ and Schmittgen T. Methods. 2000, 25, 402-408).

Western blot analysis confirmed that ErbB3 expression correlates with cell sensitivity to PM02734 (FIG. 1).

### Example 3

### Effect of PM02734 on Cell Cycle Progression in H322 Human NSCLC Cell Line

Disruption of cell cycle progression is associated with cell growth inhibition by some chemotherapeutic agents. We investigated whether PM02734-induced cell growth inhibition is associated with disturbance of cell-cycle progression in H322 NSCLC cells.

H322 cells were plated in a 6-well plate and incubated with RPMI1640 medium with 10% fetal bovine serum at 37° C. overnight. Following cell attachment, cells were exposed to 0.5 μM of PM02734 trifluoroacetate salt at 37° C. for 0, 3, 6 and 24 hours. At the indicated time point, cells were harvested by trypsinization and fixed with cold 75% ethanol at 4° C. overnight. Cells were incubated with 1 μg/ml of propidium iodide and 5 μg/ml of RNase I at room temperature for 3 hours. The cell-cycle distribution and apoptotic cells (Sub-G0/G1) were measured by FACS analysis (BD Biosciences, San Joes, Calif.).

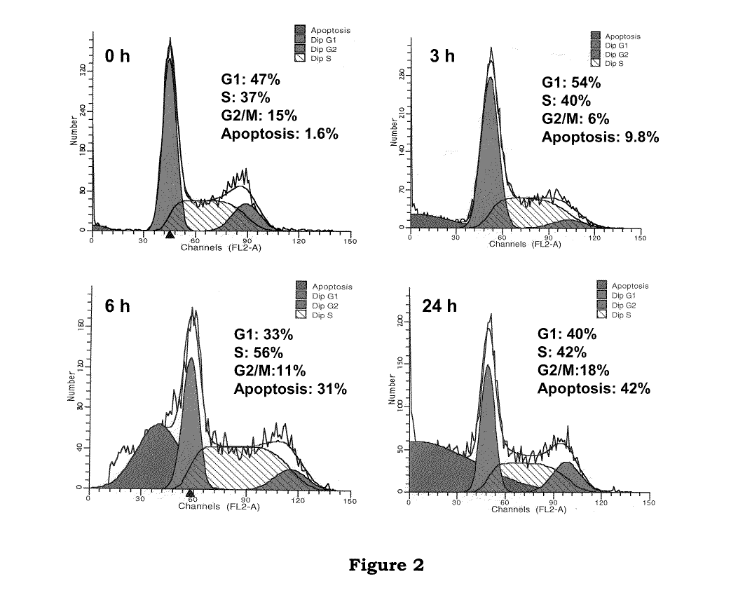

PM02734 caused cell cycle arrest at S-phase and apoptotic cell death in a time-dependent manner in PM02734-sensitive H322 cells, in contrast with Erlotinib, which caused G1/S phase arrest (Ling et al., Mol Pharmacol, 2007, 72: 248-256) (FIG. 2).

### Example 4

### Determination of Possible Synergism Between PM02734 and Erlotinib in Human NSCLC Cell Lines

Based on the observation that ErbB3 is a major determinant of cell sensitivity to PM02734, we tried to determine whether the combination of PM02734 and erlotinib, a potent EGFR tyrosine kinase inhibitor, could be synergistic.

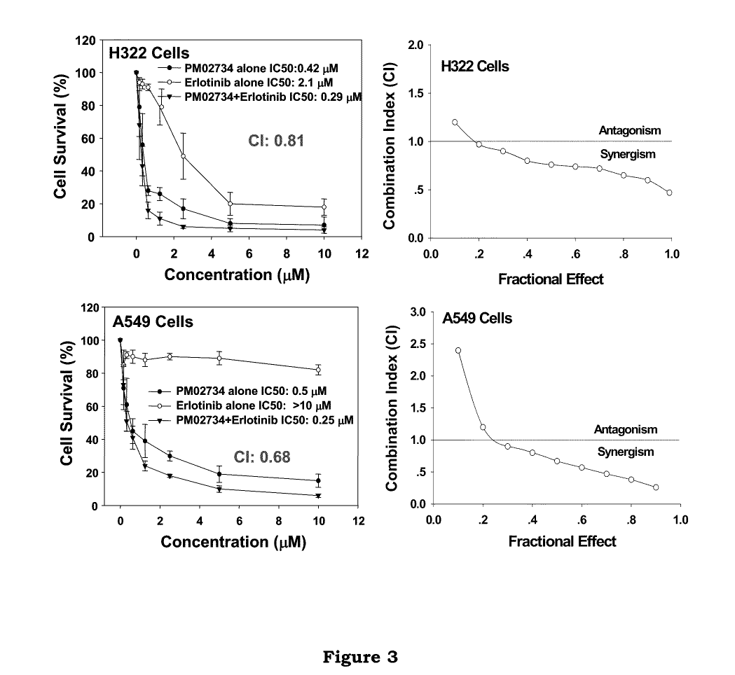

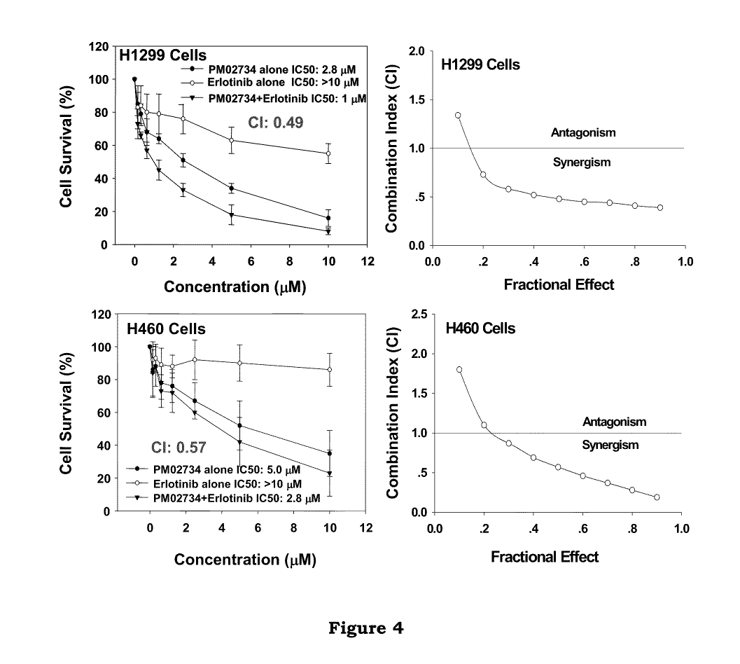

H322, A549, H1299 and H460 cells were plated in 96-well plates as described above. After overnight incubation at 37° C., attached cells were treated with various concentrations of PM02734 trifluoroacetate salt or erlotinib alone or combination of both compounds using concomitant or sequential schedules at various concentrations of PM02734 and erlotinib with 1:1 molar ratio at 37° C. for 72 h. Cell survival fractions were determined by MTT assay as described above, and the combinational effects were analyzed by the median effect method of Chou and Talalay (Chou and Talalay, Adv. Enzyme Regul. 1984, 22, 27-55). The synergistic effects (CI index) were analyzed by software CalcuSyn. CI values: <1, or >1 represent synergism or antagonism. The combination of PM02734 and erlotinib was synergistic in all cell lines irrespective of their sensibility to either agent alone, with combination indexes ranging between 0.49 and 0.81 (FIGS. 3 and 4).

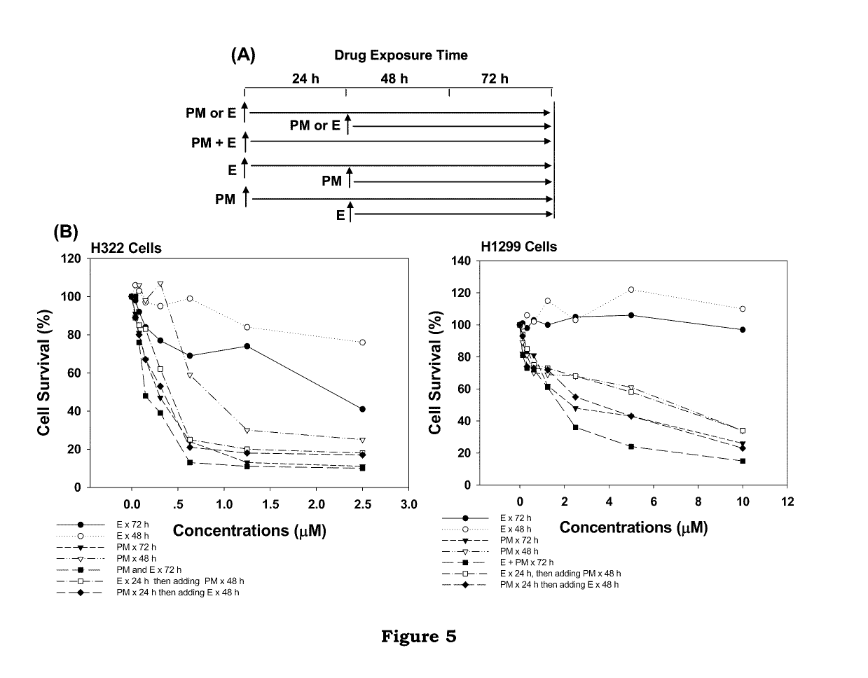

In addition, we investigated the effect of different exposure schedules of PM02734 trifluoroacetate salt (PM) and erlotinib (E) on cell growth. H322 or H1299 cells were plated in a 96-well plate overnight at 37° C., and then exposed to various concentrations of PM02734 or erlotinib alone or to the combination of both agents with different schedules as described in FIG. 5A. After schematic exposure, the cell survival was determined by a MTT assay as described above. The concurrent exposure to both agents for 72 h was more effective in inhibiting cell growth than the sequential schedules: PM02734 followed by erlotinib, or erlotinib followed by PM02734 (FIG. 5B).

### Example 5

### Effect of PM02734 on the Activation of EGFR and its Related Pathways in H322 Cell Line

We investigated whether PM02734-induced suppression of EGFR activation and its related downstream pathways is associated with cell response to this agent. To further explore the mechanism of synergism, we examined the combinational effect of PM02734 and erlotinib on the EGFR pathway.

Thus, we selected H322 sensitive cell line as model. H322 cells were starved by incubation with serum free medium in the presence of 0.5 μM of erlotinib or 0.5 μM of PM02734 trifluoroacetate salt at 37° C. for 24 h. Following stimulation with 100 μg/ml of EGF (Epidermal growth factor) for 5 min, cells were harvested and lysed with lysis buffer at 0° C. for 10 min. Equal amounts (30 μg of total protein) of lysates were subject to a 10% SDS-PAGE. After transfer to membrane, total EGFR family receptors and phosphorylated EGFR family receptors, total PI3/AKT and phosphorylated PI3/AKT, as well as total ERK1/2 and phosphorylated ERK1/2 were detected by Western blot analysis with the corresponding antibodies. β-Actin was used as a loading control. Results were quantified using a laser scanning densitometer (Kodak Image Station 440, New Haven, Conn.).

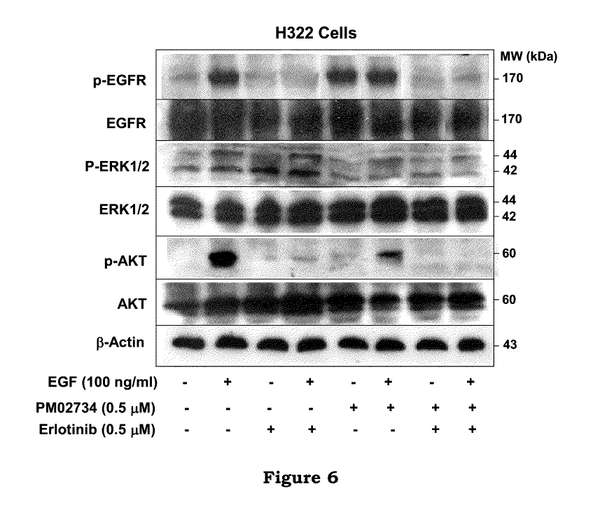

The combinational effect of PM02734 and erlotinib on the EGFR pathway was more effective in inhibiting PI3/AKT phosphorylation than either single agent alone in H322 cells (FIG. 6).

### Example 6

### In Vivo Effects of the Combination of PM02734 and Erlotinib

PM02734 was administered in its trifluoroacetate salt using water for injection as vehicle. Erlotinib was administered using sterile water as vehicle.

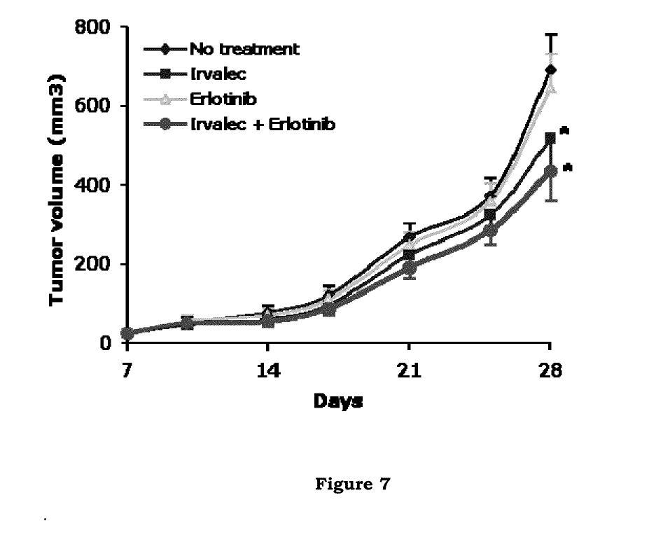

Nude mice (NUR-NU-F-M, female, 5-6 weeks old, Taconic Farm, Germantown N.Y.) were subcutaneously implanted with the human A549 NSCLC cell line (4.5×10⁶ cells/mouse). Seven days later, mice were randomly divided into 4 groups with 5 mice per group. One group of mice received the combination treatment (PM02734: 0.1 mg/kg×3 times per week (on days 1, 3, and 5) for 2 weeks, i.v.; erlotinib: 50 mg/kg×5 times per week (on days 1, 2, 3, 4, and 5) for 2 weeks, p.o.). In this group erlotinib was given first followed two hours later by the administration of PM02734. The other groups were treated with PM02734 alone (0.1 mg/kg×3 times per week (on days 1, 3, and 5) for 2 weeks, i.v.), erlotinib alone (50 mg/kg×5 times per week (on days 1, 2, 3, 4, and 5) for 2 weeks, p.o.) or without treatment. Tumor size was measured twice a week. Tumor volume was calculated using the following equation: Tumor volume (mm³)=0.5×longer diameter×(shorter diameter)² in millimeter. The experiment was terminated when ≧50% mice had tumors larger than 15 mm (i.e., the longer diameter>15 mm). As shown in FIG. 7, a statistically significant reduction of tumor volume was observed with the treatment administering PM02734 alone (% treated vs. control tumor volume ratio: % T/C=74%, p<0.001) and with the treatment administering PM02734 in combination with erlotinib (% T/C=62%, p<0.001).

In the orthotopic model, nude mice (NUR-NU-F-M, female, 5-6 weeks old, Taconic Farm, Germantown N.Y.) were intravenously inoculated with the human A549 NSCLC cell line (4.2×10⁶ cells/mouse). Under these experimental conditions i.v. inoculated A549 cells grow only in the lungs but not in other organs, and the survival of mice is directly related to the tumor burden. Nine days after tumor inoculation mice were randomly divided into 4 groups with 7 mice in the combination group and 5 mice in the other groups. The dose and treatment schedule was the same as those used in the subcutaneous xenograft model. Mice were observed daily and survival or % Increased Life Span was determined.

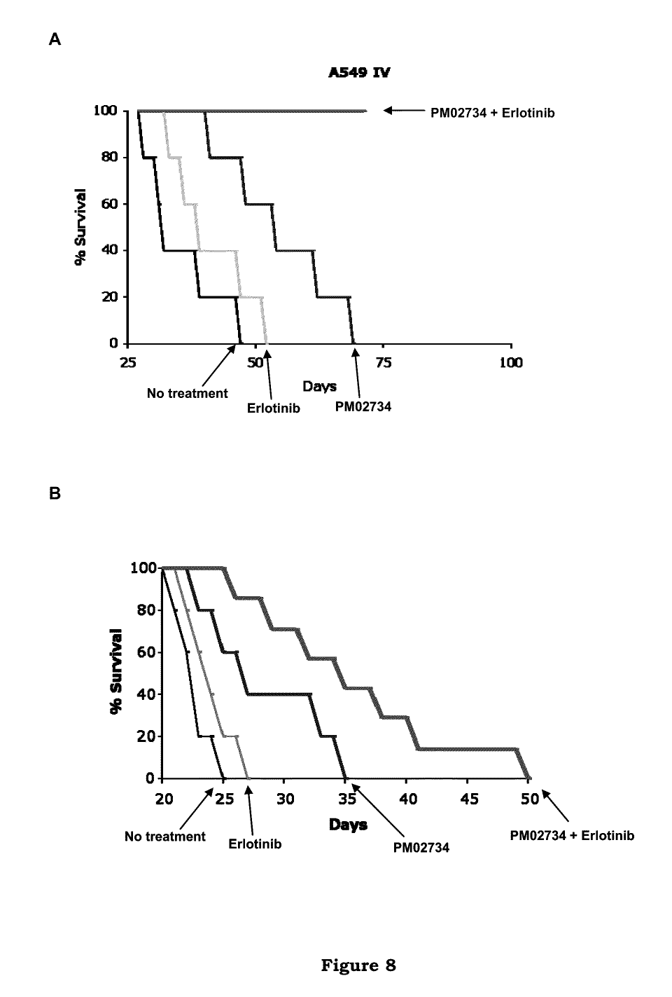

As shown in FIG. 8, in the two experiments with different number of cells inoculated a statistically significant increase in life span (or survival) was observed in the group treated with the combination of PM02734 with erlotinib vs. the other groups (no treatment, PM02734 alone or erlotinib alone) (p=0.0003 for FIG. 8A; p=0.002 for FIG. 8B).

These findings in respect of erlotinib can be extended to other EGFR tyrosine kinase inhibitors. For example, the present invention provides a combination of PM02734 with EGFR tyrosine kinase inhibitors such as those disclosed in U.S. Pat. No. 5,457,105, U.S. Pat. No. 5,770,599, U.S. Pat. No. 5,747,498, U.S. Pat. No. 6,344,455, U.S. Pat. No. 6,391,874, U.S. Pat. No. 6,713,485, U.S. Pat. No. 6,727,256, U.S. Pat. No. 6,900,221, U.S. Pat. No. 7,157,466, U.S. Pat. No. 4,943,533, U.S. Pat. No. 5,558,864, U.S. Pat. No. 5,891,996, and U.S. Pat. No. 6,235,883, and specially with erlotinib, gefitinib, canertinib, lapatinib, cetuximab, matuzumab, zalutumumab, and panitumumab.

1-24. (canceled)

25. A method of treating cancer comprising administering to a patient in need of such treatment a therapeutically effective amount of PM02734, or a pharmaceutically acceptable salt thereof, and a therapeutically effective amount of an EGFR tyrosine kinase inhibitor, or a pharmaceutically acceptable salt thereof.

26. A method of increasing the therapeutic efficacy of an EGFR tyrosine kinase inhibitor in the treatment of cancer, which comprises administering to a patient in need thereof, the EGFR tyrosine kinase inhibitor and a therapeutically effective amount of PM02734, or a pharmaceutically acceptable salt thereof.

27. The method according to claim 25, wherein the EGFR tyrosine kinase inhibitor is selected from erlotinib, gefitinib, canertinib, lapatinib, cetuximab, matuzumab, zalutumumab, and panitumumab, or a pharmaceutically acceptable salt thereof.

28. The method according to claim 26, wherein the EGFR tyrosine kinase inhibitor is selected from erlotinib, gefitinib, canertinib, lapatinib, cetuximab, matuzumab, zalutumumab, and panitumumab, or a pharmaceutically acceptable salt thereof.

29. The method according to claim 27, wherein the cancer to be treated is selected from leukemia, melanoma, breast cancer, colon cancer, colorectal cancer, ovarian cancer, gynecological cancer, renal cancer, head and neck carcinoma, esophageal cancer, pancreatic cancer, lung cancer, cervix cancer, liver cancer, and prostate cancer.

30. The method according to claim 28, wherein the cancer to be treated is selected from leukemia, melanoma, breast cancer, colon cancer, colorectal cancer, ovarian cancer, gynecological cancer, renal cancer, head and neck carcinoma, esophageal cancer, pancreatic cancer, lung cancer, cervix cancer, liver cancer, and prostate cancer.

31. The method according to claim 29, wherein PM02734, or a pharmaceutically acceptable salt thereof, and the EGFR tyrosine kinase inhibitor, or a pharmaceutically acceptable salt thereof, form part of the same composition.

32. The method according to claim 29, wherein PM02734, or a pharmaceutically acceptable salt thereof, and the EGFR tyrosine kinase inhibitor, or a pharmaceutically acceptable salt thereof, are provided as separate compositions for administration at the same time or at different times.

33. The method according to claim 32, wherein PM02734, or a pharmaceutically acceptable salt thereof, and the EGFR tyrosine kinase inhibitor, or a pharmaceutically acceptable salt thereof, are provided as separate compositions for administration at different times.

34. The method according to claim 30, wherein PM02734, or a pharmaceutically acceptable salt thereof, and the EGFR tyrosine kinase inhibitor, or a pharmaceutically acceptable salt thereof, form part of the same composition.

35. The method according to claim 30, wherein PM02734, or a pharmaceutically acceptable salt thereof, and the EGFR tyrosine kinase inhibitor, or a pharmaceutically acceptable salt thereof, are provided as separate compositions for administration at the same time or at different times.

36. The method according to claim 35, wherein PM02734, or a pharmaceutically acceptable salt thereof, and the EGFR tyrosine kinase inhibitor, or a pharmaceutically acceptable salt thereof, are provided as separate compositions for administration at different times.

37. A kit for administering PM02734, or a pharmaceutically acceptable salt thereof in combination with an EGFR tyrosine kinase inhibitor, or a pharmaceutically acceptable salt thereof, comprising a dosage form of PM02734 or a pharmaceutically acceptable salt thereof, and/or a dosage form of an EGFR tyrosine kinase inhibitor, or a pharmaceutically acceptable salt thereof, and printed instructions for administering both drugs in combination.


SEQUENCE LISTING (8 sequences)

SEQ ID NO: 1 - DNA, 20 bp, Artificial Sequence
  synthetic primer
ccacctgtgccatccaaact

SEQ ID NO: 2 - DNA, 18 bp, Artificial Sequence
  synthetic primer
ggcgatggacgggatctt

SEQ ID NO: 3 - DNA, 18 bp, Artificial Sequence
  synthetic primer
agccttgcccatcaactg

SEQ ID NO: 4 - DNA, 18 bp, Artificial Sequence
  synthetic primer
aatgccaaccaccgcaga

SEQ ID NO: 5 - DNA, 24 bp, Artificial Sequence
  synthetic primer
tcctggccgccccacatgcacaac

SEQ ID NO: 6 - DNA, 20 bp, Artificial Sequence
  synthetic primer
gtcacatttgccctctgcca

SEQ ID NO: 7 - DNA, 20 bp, Artificial Sequence
  synthetic primer
catgggtcagaaggattcct

SEQ ID NO: 8 - DNA, 20 bp, Artificial Sequence
  synthetic primer
cattgtagaaggtgtggtgc